(19) United States
(12) Patent Application Publication
(10) Pub. No.: US 2013/0077048 A1
(43) Pub. Date: Mar. 28, 2013

(54) INTEGRATED FIBER OPTIC OPHTHALMIC INTRAOCULAR SURGICAL DEVICE WITH CAMERA

(71) Applicant: Ram Srikanth Mirlay, Bangalore (IN)

(72) Inventor: Ram Srikanth Mirlay, Bangalore (IN)

(21) Appl. No.: 13/702,914

(22) PCT Filed: Mar. 7, 2011
(86) PCT No.: PCT/IN11/00143

§ 371 Date: Dec. 7, 2012

(30) Foreign Application Priority Data
Jun. 10, 2010 (IN) 1607/CHE/2010

(51) Int. Cl. A61B 3/14

(52) U.S. Cl. CPC A61B 3/145
USPC 351/206

(57) ABSTRACT

A fiber optic ophthalmic surgical microscope with camera assembly comprises a fiber optic cable (102), a micro lens unit (103), a surgical instrument attached with the fiber optic cable (102), a signal splitter (105), a surgical operating microscope (106), a switch-over mechanism (111) and at least a device for viewing the images. The surgical instrument includes a chopper, a dialer, sinsky's hooks, a manipulator, micro forceps, a coaxial irrigation and aspiration (Infusion Aspiration) canula, bimanual Infusion Aspiration canula or combination thereof. The switch-over mechanism is a button. The button is provided on the signal splitter. The device for viewing the images is selected from a group comprising TV monitor and VCR.

### BACKGROUND

1. Technical field

The embodiments herein generally relate to an ophthalmic surgical device and particularly to an ophthalmic microscope with a camera for capturing and viewing still and live images or videos from inside the mammalian eye externally on a TV or monitor or during surgery. The embodiments herein more particularly relate to an ophthalmic surgical microscope with a fiber optic camera.

2. Description of the Related Art

A camera system provides a doctor/surgeon to view the various internal parts of the eye. The field of surgical microscopy encompasses a wide variety of applications, along with the various operating equipment used for these applications.

A surgical operating microscope is a standard tool used in the operating room. The ophthalmic microscope is typically a binocular head that is angled. Video microscopy is also an added feature in a surgical operating microscope. Many of the models have optional beam splitters to split the image to an optional c-mount where a CCD camera can be attached. These CCD cameras have NTSC or PAL video output signals to be taken to a CCTV viewing/display monitor. This video microscopy equipment makes an excellent options package as it allows multiple simultaneous viewing by all physicians and assistants in the operating room.

Ophthalmoscope is a lighted instrument and is one of the most important tools of the physician, used to examine the interior of the eye, including the lens, retina and optic nerve. The common clinical ophthalmoscope consists of a concave mirror and a battery-powered light (contained within the handle). The operator looks through a single monocular eye piece into the patient's eye. The ophthalmoscope is equipped with a rotating disc of lenses to permit the eye be examined at different depths and magnifications. This may be enhanced by drugs that dilate the pupil and enlarge the opening into the structures within the eye. But it is difficult to view the internal parts hidden behind the iris, which are opaque in nature.

Hence there is a need to provide a camera device to provide a unique way of viewing the internal parts of the eye during eye surgery. Also there is a need to provide a view of the normally hidden structures behind the iris during an eye surgery.

The above mentioned shortcomings, disadvantages and problems are addressed herein and which will be understood by reading and studying the following specification.

### OBJECTS OF THE EMBODIMENTS

An objective of the embodiments herein is to provide an ophthalmic surgical microscope with a fiber optic camera to view the essential and vital internal structures of the eye during an eye surgery.

Another objective of the embodiments herein is to provide an ophthalmic surgical microscope with a fiber optic camera to view of the internal structures, normally hidden behind the iris, which are opaque in nature.

Yet another objective of the embodiments herein is to provide an ophthalmic surgical microscope with a fiber optic camera having a switch-over mechanism so that the surgeon switches from a fiber optic cable view to a direct microscopic view, or vice versa, while performing an eye surgery.

Yet another objective of the embodiments herein is to provide a fiber optic camera that is slim, long and can bend gently, and provide easy entry and maneuverability inside the eye during surgery.

Yet another objective of the embodiments herein is to provide a fiber optic camera to which different surgical instruments are attached.

These and other objects and advantages of the embodiments herein will become readily apparent from the following detailed description taken in conjunction with the accompanying drawings.

These and other objects and advantages of the embodiments herein will become readily apparent from the following detailed description taken in conjunction with the accompanying drawings.

### SUMMARY

The various embodiments herein provide a fiber optic image or light capture device in an ophthalmic surgical microscope to capture and provide still and live, video images from inside the mammalian eye and to display externally on a TV monitor or an operating microscope during surgery. The device is attached to an operating Microscope. The device is mated to various surgical instruments to create newer devices.

According to one embodiment embodiments herein, a fiber optic intraocular instrument with camera comprising a fiber optic cable, a micro lens unit at one end of the fiber optic cable, at least a surgical instrument attached with the one end of the fiber optic cable, a camera connected with an another end of the fiber optic cable, a signal splitter connected with the camera, a surgical operating microscope connected with the signal splitter, a switch-over mechanism connected with the surgical operating microscope and at least a device connected with the switch over mechanism for viewing the images. The fiber optic has a diameter of 0.5 to 2 mm. The surgical instrument is selected from a group comprising of a chopper, a dialer, sinskey hooks, a manipulator, a micro forceps, a coaxial irrigation, aspiration (Infusion Aspiration) canula, bimanual Infusion Aspiration canula and combination thereof. The switch-over mechanism is a retractable LCD screen. The retractable LCD in placed inside an eyepiece of the surgical operating microscope. The switch-over mechanism is a button. The button is present on the signal splitter. The button is connected with the retractable. LCD. The device for viewing the images is selected from a group comprising of TV monitor and VCR.

According to one embodiment embodiments herein, the fiber optic camera assembly enables a doctor or a surgeon to view the essential and vital internal parts of the eye during an eye surgery. The camera assembly is provided to display a unique view of the structures that are normally hidden behind the iris, mainly, the equatorial region of the mammalian eye lens and angle of anterior chamber.

According to one embodiment herein, the camera assembly has a high optical grade fiber optic cable with a diameter of 0.5 to 2 mm. A micro lens unit is fixed at one distal end of the optical fiber cable and a camera is fixed at another end of the optical fiber cable. The range of focus of lenses is between 02 mm to 10 mm. The camera is capable of capturing still and live images of the internal parts of the eye. A beam splitter complex is attached which transfers the images to a TV or a monitor or a LCD screen or to the surgical operating microscope. The tip of fiber optic camera is slim, long and can be bent gently, to provide an easy entry and maneuverability inside the eye during surgery. The beam splitter unit transfers the visual images to a TV or a monitor or a LCD screen or to a display screen in a surgical operating microscope.

The camera assembly has a switch-over mechanism to help a surgeon to change the views simultaneously while conducting a surgery. The switch-over mechanism is a retractable LCD screen. The retractable LCD screen is placed in an optical path of one of the eyepiece of the surgical operating microscope. The surgeon gets two views, one, from the fiber optic camera and second, directly from the surgical operating microscope. When not required, the surgeon may take out the LCD screen. The captured images acquired from the fiber optic cable are displayed on TV monitor or an operating microscopic.

According to one embodiment, an illumination light source (halogen) is connected to a portion of the fiber optic Bundle as an attachment. The illumination light source is selected from a group comprising of a halogen, xenon arc, bulb filaments and combination thereof. The light source is separately present and connected with the fiber optic cable with an adaptor.

According to one embodiment, pluralities of surgical instruments that are used inside the eye are attached with the fiber optic camera to create newer devices used for a cataract surgery using a phaco emulsification technique. The pluralities of surgical instrument include chopper, dialer, sinskey hooks, manipulator, micro forceps, coaxial irrigation and aspiration (I.A) canula, bimanual I.A canula and various combinations and modifications of the above. A common feature of all these instruments is that they are slim and long and bent gently for easy entry and maneuverability inside the eye during surgery.

The plurality of surgical instruments is attached with the fiber optic by an adhesive, wire, clasps, band or any other method. The plurality of surgical instruments is welded with the fiber optic.

According to one embodiment herein, the fiber optic camera assembly goes inside the eye through an incision made in the cornea of the eye and helps in viewing the inner parts of the eye. The images are displayed on the LCD screen present inside one of the eyepiece. The images are also displayed on a TV screen or monitor. The video is also recorded using a VCR.

According to one embodiment herein, the fiber optic cable has a finger grip pad or surface to hold the fiber optic.

The fiber optic camera (F.O.C) assembly has a flexible tube with a profile which is similar to each of the surgical instruments used during a surgery. Each of the above mentioned instruments is joined to FOC assembly using adhesives, wires, clasps or any other method to form a new instrument to facilitate critical visibility to the surgeon during the use of this innovation. The plurality of surgical instruments is welded with the fiber optic.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a block diagram of an ophthalmic surgical microscope with fiber optic camera, according to one embodiment herein.

FIG. 2 shows a side view of the fiber optic cable attached with an aspiration canula, according to one embodiment herein.

FIG. 3 shows a sectional view of a fiber optic cable attached with a chopper, according to another embodiment herein.

FIG. 4 shows a sectional view of a fiber optic intraocular instrument with camera showing the insertion of fiber optic cable into an eye, according to an embodiment herein.

Although specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments herein are described in sufficient detail to enable those skilled in the art to practice the embodiments herein and it is to be understood that the logical, mechanical and other changes may be made without departing from the scope of the embodiments herein. The following detailed description is therefore not to be taken in a limiting sense.

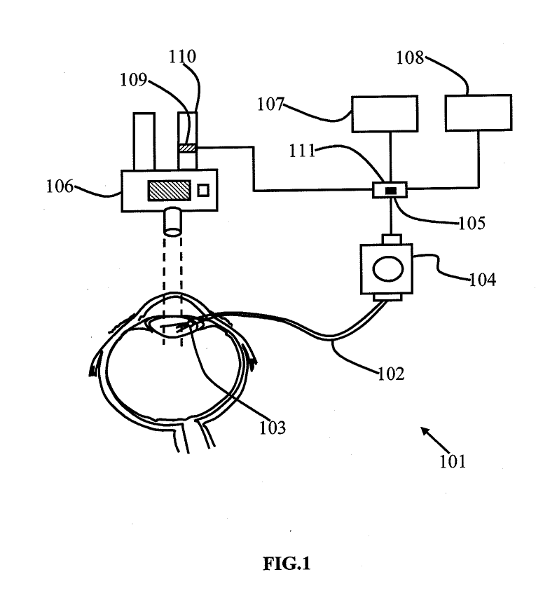

FIG. 1 shows a block diagram of fiber optic ophthalmic intraocular, instrument with camera, according to one embodiment herein. With respect to FIG. 1, fiber optic intraocular instrument with camera 101 comprises fiber optic cable 102 with a micro lens unit 103 at one end. Fiber optic cable 102 is a high optical grade fiber optic of 0.5 to 2 mm diameter. Tip of fiber optic cable 102 is slim, long and can be bent gently to provide an easy entry and maneuverability inside an eye during surgery. Range of focus of lenses in micro lens unit 103 is between 02 mm to 10 mm. Camera 104 is attached at the other end of fiber optic cable 102. Camera 104 is capable of capturing still and live images of the internal parts of the eye. Signal splitter 105 is connected with camera 104 and splits the signal coming from camera 104 to surgical operating microscope 106, TV screen 107 and VCR 108. Surgical operating microscope 106, TV screen 107 and VCR 108 are connected with signal splitter 105. Retractable LCD screen 109 is placed in one of the eyepiece 110 of surgical operating microscope 106. Retractable LCD screen 109 forms a switch-over mechanism to switch over to different views. Retractable LCD screen 109 is taken out when not required by surgeon.

According to one embodiment herein, switch 111 on signal splitter 105 controls the image viewing on surgical operating microscope 106, TV screen 107, VCR 108 and retractable LCD screen 109. Switch 111 is connected with retractable LCD screen 109.

According to various embodiments herein, different surgical instrument can also be attached with the fiber optic camera.

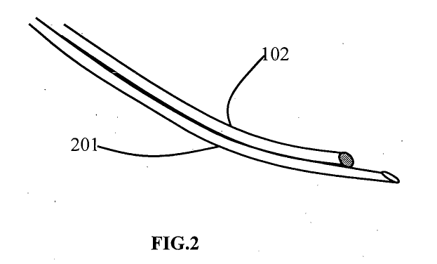

FIG. 2 shows a side view of the fiber optic cable attached with an aspiration canula, according to one embodiment herein. With respect to FIG. 2, aspiration canula 201 is attached with fiber optic cable 102.

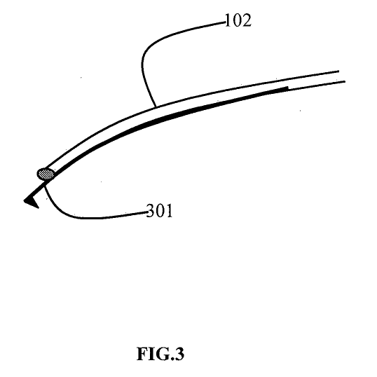

FIG. 3 shows a sectional view of a fiber optic cable attached with a chopper, according to an embodiment herein. With respect to FIG. 3, chopper 301 is attached with fiber optic cable 102.

According to one embodiment, pluralities of surgical instruments that are used inside the eye are attached with the fiber optic camera to create newer devices used for a cataract surgery using a phaco emulsification technique. The pluralities of surgical instrument include chopper, dialer, sinsley hooks, manipulator, micro forceps, coaxial irrigation and aspiration (Infusion Aspiration) canula, bimanual Infusion Aspiration canula and various combinations and modifications of the above. A common feature of all these instruments is that they are slim and long and bent gently for easy entry and maneuverability inside the eye during surgery.

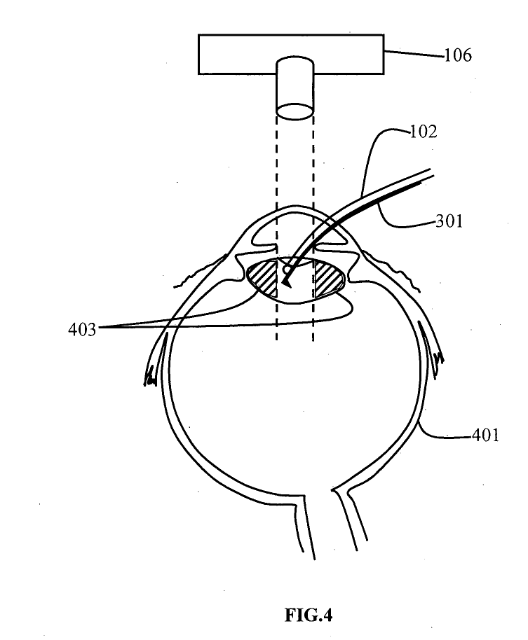

FIG. 4 shows a sectional view of a fiber optic intraocular instrument with camera showing the insertion of fiber optic cable into an eye, according to an embodiment herein. With respect to FIG. 4, fiber optic cable 102 is attached with chopper 301. Fiber optic 102 attached with chopper 301 is inserted into eye 401. Area 403 inside eye 401 is visible using fiber optic intraocular instrument with camera assembly 101. Area 403 of eye 401 is not visible using surgical operating microscope 106.

The embodiments herein provide a fiber optic image or a light capture device to capture and provide still and live, video images from inside the mammalian eye and to display externally on a TV monitor or an operating microscope during surgery. The device is attached to an operating Microscope. The device is mated to various surgical instruments to create newer devices.

According to one embodiment herein, a camera assembly is provided to enable a doctor or surgeon to view the essential and vital internal parts of the eye. The camera assembly is provided to display a unique view of the structures that are normally hidden behind the iris.

According to one embodiment herein, a beam splitter unit is attached to the camera to transfer the images to a TV or a monitor or a LCD screen or to the surgical operating microscope. The beam splitter unit is provided so that the visual images are transferred to a TV monitor or LCD screen or to a display screen in a surgical operating microscope.

According to one embodiment herein, a switch-over mechanism is attached to the fiber optic camera system to help the surgeon to change the views simultaneously. While performing surgery, the surgeon is able to see only one view in general, i.e. fiber optic cable view or TV or monitor view or operating microscopic view or an LCD view. With this attachment, he can switch over the views simultaneously. The switch-over mechanism is a retractable LCD screen, according to one embodiment herein. To change the LCD view, the LCD screen can be taken out. The switch-over mechanism is a switch or button present on the signal splitter to switch to various views, according to another embodiment herein. The various views comprise a view on TV monitor, a view on LCD screen in an eyepiece of surgical operating microscope, and a view on the VCR. A video of the surgery can also be recorded using the VCR. The captured images acquired from the fiber optic cable are displayed on TV monitor or an operating microscopic or VCR or LCD screen.

According to one embodiment, an illumination light source is connected to a portion of the fiber optic Bundle as an attachment. The illumination light source is selected from a group comprising of a halogen, xenon arc, bulb filaments and combination thereof. The light source is separately present and connected with the fiber optic cable with an adaptor.

The fiber optic camera (F.O.C) assembly has a flexible tube with a profile which is similar to each of the surgical instruments used during a surgery. Each of the above mentioned instruments is joined to FOC assembly using adhesives, wires, band, clasps, and welded together or any other method to form a new instrument to facilitate critical visibility to the surgeon. Thus a fiber optic camera system provides a doctor/surgeon to view the internal parts of the eye while performing surgery. The parts that are hidden behind the iris and are difficult to view during a surgery are easily seen by the surgeon with the help of the camera system.

The various surgical instruments which can be attached are dialer, sinsley hooks, manipulator, micro forceps, coaxial irrigation and aspiration (I.A) canula, bimanual I.A canula and various combinations and modifications of the above. A common feature of all these instruments which can be attributed is that they are slim, long and bent gently to provide an easy entry and maneuverability inside the eye during a surgery. The fiber optic camera tip also has same profile as that of each surgical instrument to be attached thereby providing a critical visibility of the camera to the surgeon during its use. The above mentioned instruments can be attached by various means, for example, adhesives, wires, or any other suitable method.

According to one embodiment herein, the integrated fiber optic ophthalmic intraocular surgical device is a one piece i.e. the surgical instruments are made up of the same material as that of the fiber optic cable by extending a portion of the fiber optic cable. The one piece is a transparent surgical instrument that helps to see the area under the surgical instrument while inserted into an eye. According to an embodiment herein, the fiber optic chopper device includes a fiber optic cable and a chopper made by extending a portion of the fiber optic cable. According to another embodiment herein, the fiber optic aspiration device includes a fiber optic cable and an aspiration canula. The aspiration canula is made up of the same material as that of fiber optic cable. According to one embodiment herein, fiber optic infusion canula includes fiber optic cable and an infusion canula made up of the same material as that of fiber optic cable. The surgical instruments that can be made along with fiber optic cable as an extension are selected from a group comprising dialer, sinsley hooks, manipulator, micro forceps, coaxial irrigation and aspiration (I.A) canula, bimanual I.A canula and various combinations and modifications of the above.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of-the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A fibre optic ophthalmic surgical microscope with camera assembly comprising:
a fibre optic cable;
a micro lens unit mounted at one end of the fibre optic cable;
a camera attached to another end of the fiber optic cable;
a surgical instrument attached to the fiber optic cable;
a signal splitter communicatively connected to the camera;
a surgical microscope communicatively connected to the signal splitter;
a display screen communicatively connected to the signal splitter;
a signal recording unit connected to the signal splitter;
a switch-over mechanism connected with the surgical microscope;
wherein the camera is arranged to provide a view of an area behind iris in an eye which is normally not visible during a surgery so that a surgeon is provided with both a direct view of an eye and an indirect view of the area behind iris on a monitor during an eye surgery.

2. The assembly according to claim 1, wherein the fiber optic has a diameter of 0.5 to 2 mm.

3. The assembly according to claim 1, where in the at least a surgical instrument is selected from a group comprising of a chopper, a dialer, sinsky hooks, a manipulator, a micro forceps, a coaxial irrigation, aspiration (Infusion Aspiration) canula, bimanual Infusion Aspiration canula and combination thereof.

4. The assembly according to claim 1, wherein the switch-over mechanism is a button.

5. The assembly according to claim 6, wherein the button is present on the signal splitter.

6. The assembly according to claim 1, wherein the signal splitter is provided to pass an output of the camera to an eye piece of the surgical microscope, to the display screen for projecting an image and to the signal recording unit for storing and recording the image.

7. The assembly according to claim 1, wherein the display device is selected from a group comprising a television receiver, ALCD monitor, LED monitor and a plasma display device.

8. The assembly according to claim 1, wherein the fiber optic cable and the surgical instrument are formed integrally as a single piece.

9. The assembly according to claim 1, wherein the surgical instrument is made up of a fiber optic material to enable surgical operation in the eye and to see an area under the surgical instrument simultaneously during insertion into eye.

10. The assembly according to claim 1, wherein the fiber optic cable and the surgical instrument are formed integrally as a single piece by extending a potion of the fiber optic cable.
